(19) United States
(12) Patent Application Publication
(10) Pub. No.: US 2003/0195482 A1
(43) Pub. Date: Oct. 16, 2003

(54) Pneumatic medical system

(76) Inventor: Joseph P. Schultz, Phoenix, AZ (US)

Correspondence Address: STONEMAN LAW OFFICES, LTD, 3113 NORTH 3RD STREET, PHOENIX, AZ, 85012, US

(21) Appl. No.: 10/413,080

(22) Filed: Apr. 14, 2003

Related U.S. Application Data
(63) Continuation-in-part of application No. 10/067,459, filed on Feb. 4, 2002, now abandoned.
Application No. 10/067,459 is a continuation of application No. 60/266,110, filed on Feb. 2, 2001.

(51) Int. Cl. A61M 1/00

(52) U.S. Cl. 604/317

(57) ABSTRACT

A pneumatic medical system, which includes a collection canister with a lid. Integrally attached to the lid are inlet and outlet ports, a thumb valve to regulate the level of suction or pressure in the system, a clip to hold a loop of catheter, an endotracheal tube adaptor, and a single hole for a single-suction catheter. The suction catheter has telescoping lengths and sizes of catheter attached so that the user can increase the diameter of catheter to be used by removing smaller diameter catheters. A smaller diameter catheter can be removed by pulling the smaller diameter catheter through the telescoping nested attachment with the larger diameter catheter. If the canister becomes full or clogged, it can be easily cleared by tipping the canister and pouring the contents through the thumb valve. The system requires only one hand and is easy to hold and manipulate. A disposable and pre-filled irrigation system is also disclosed.

### CROSS REFERENCE TO RELATED APPLICATIONS

The present application is a continuation-in-part of related Utility application Ser. No. 10/067,459, filed Feb. 4, 2002, entitled “PNEUMATIC MEDICAL SYSTEM”, and Applicant's prior U.S. Provisional Application Serial No. 60/266,110, filed Feb. 2, 2001, entitled “PNEUMATIC MEDICAL SYSTEM”, the contents of which are herein incorporated by reference and are not admitted to be prior art with respect to the present invention by its mention in this cross-reference section.

### BACKGROUND

Pneumatic systems have a variety of uses in medical settings. Newborn babies are sometimes “suctioned” when they are born. Suction devices, such as bulb syringes, suction catheters or DeLee traps, are used to remove fluid, meconium and other secretions from the baby's mouth, nose, lungs and stomach. Suction devices can be attached to sterile canisters for collecting specimens from suctioned material for later laboratory analysis. This is used on both pediatric and adult patients. Sometimes the traps are used in conjunction with adaptors to other suction devices. Suction is also used in many other medical applications.

These devices, bulb syringes, flexible suction catheters and DeLee traps, have advantages and disadvantages. Bulb syringes are easy to use, but have limited capacity. Suction catheters are inexpensive, disposable, and effective. However, they might not be of the correct length, suction strength, or size to effectively suction some material. For example, a suction catheter that is of optimal size for a newborn would be too small for an older child or adult. Suction catheters may be difficult to control. For example, a long thin catheter may bend or kink, decreasing its ability to perform suction. It may also be difficult to control the power of the suction using a suction catheter. Especially when using such an instrument with a newborn, it is desirable to be able to increase or decrease the amount of suction being administered to the patient.

DeLee traps consist essentially of two catheter tubes inserted through a lid on a measured canister. One tube is used to provide suction, and the other tube is used to insert into the patient's nose or mouth to remove fluids. Early suction devices, including the DeLee trap, were designed to use oral suction. Today, concerns about communicable diseases prevent medical personnel from engaging in oral suction. Originally, the DeLee trap was used to measure the 20 cc volume of fluid that, if aspirated from the stomach, would indicate that there might be a gastric outlet obstruction. This volume of fluid may be predictive of a poor outcome for the baby. While this measurement is of less significance today, with other means for assessing gastric outlet obstructions, the DeLee trap is still commonly used for suctioning and specimen collection. DeLee suction devices have been modified to use a thumb valve to control the level of suction when attached to a mechanical suction source.

DeLee traps can become clogged. Usually, in a DeLee trap, the inlet tube is longer and extends further into the specimen canister than the outlet tube. As the canister fills with specimen fluid, the inlet tube may become blocked, leaving the inlet tube with a volume space that may be difficult for the vacuum to overcome. Once clogged, it can be very difficult to clear a clog and reactivate the device. DeLee traps can also be very awkward to use. When operating a DeLee trap, the operator needs one hand on the distal long, flexible catheter, one hand on the canister to maintain its orientation and prevent clogging, and one finger on the flow control valve. This cumbersome arrangement is poorly suited to procedures that demand the physician and assisting medical support teams quickly transition between tasks.

Assisting in the birth of a baby, including suctioning the newborn, is one such example. Typically, the physician or midwife must assemble the DeLee trap to a vacuum source, usually with one hand holding the aggregation of tubing, and the other hand holding the canister, while simultaneously assisting with the final stages of delivery. This critical time during the delivery requires the assistance of the physician or midwife to prevent the baby from being expelled uncontrollably and possibly traumatically. The example of suctioning a newborn is just one of many instances in which specimen traps are cumbersome to use during important medical procedures.

### OBJECTS OF THE INVENTION

An object and feature of a preferred embodiment of the present invention is to fulfill the above-mentioned needs by providing a pneumatic medical system which is easy to assemble, manipulate, hold, manufacture, control, use and transport. It is also an object and feature of the preferred embodiment to provide a pneumatic system, including a vacuum aspiration system with specimen trap, that is made of fewer parts for easier manufacturing. In addition, it is an object and feature of the preferred embodiment to provide a pneumatic system, including a vacuum aspiration system with a specimen trap, that is oriented in such a way as to be easier to manually hold and control. It is also an object and feature of a preferred embodiment of this invention to provide a vacuum aspiration system which can collect fluids or other biological samples with a decreased incidence of clogging. In addition, it is an object and feature of a preferred embodiment of this invention to provide a system that is more easily cleared, should it become clogged.

It is also an object and feature of the present invention to provide a system of telescoping catheters to allow the physician more versatility in using different sizes of catheters, in connection with the suction device, or separately from it.

It is also an object and feature of this invention to provide a pneumatic medical system which can assist in the rapid administration of fluids to a patient with the use of a pressurized fluid source. It is also an object and feature of this invention to provide a pneumatic medical system that can assist in the rapid administration of fluids for irrigation purposes that may include wound irrigation with or without a splash shield. It is also an object and feature of this invention to provide a pneumatic medical system which can assist in the rapid administration of fluids to a patient with the use of an externally pressurized fluid source, which attaches to a readily available fluid reservoir, such as a bottle of sterile fluid.

It is also an object and feature of this invention to provide a pneumatic medical system which can assist in the rapid administration of fluids to a patient with the use of a pressurized fluid source, which may be continuous, or may be regulated, or may be continuously regulated. It is also an object and feature of this invention to provide a pneumatic medical system which can assist in the rapid administration of fluids to a patient with the use of a pressurized fluid source, which may be continuous, or may be regulated, or may be continuously regulated; which may be sterile or may be manufactured at a low cost, and may be disposable; which may attach to a common and readily available fluid source, such as a container of sterile fluid, such as a bottle of sterile saline; or which may have an outlet port of a fluid reservoir at or near the bottom of the reservoir, and which may have conduits or adaptors to mate with commonly available pressurized fluid sources or regulated pressurized fluid sources in medical settings, such as hospitals, which may include adaptors to connect with oxygen sources, such as oxygen tanks or oxygen regulators connected to oxygen tanks or wall sources; or which can assist in the rapid administration of fluids to a patient with the use of a pressurized fluid source, which may have a means to vary the diameter of the outflow conduit to vary the pressure of the fluid effluent, and may be adapted on the outlet end to mate with a wound irrigation shield to contain the pressurized effluent; or which can be rapidly or simply modified from a pneumatic system under positive pressure to rapidly and controllably administer fluid to a pneumatic system under negative pressure for use in containing suctioned specimens with a trap; and this might be accomplished by distraction or repositioning of an outflow tube to simplify stocking and promote recycling in medical settings.

It is also an object and feature of this invention to provide a multi-purpose pneumatic medical system that could be rapidly changed from catheters of varying dimensions for various functions, that might be constructed of telescopic or fixed tubing with a minimal amount of parts or assembly required.

It is a further object and feature of the present invention to provide such a system that is pre-filled with an irrigation fluid and is economical as a single use device.

Additional objects and features of this invention will become clear with the following description and claims.

### SUMMARY OF THE INVENTION

In accordance with a preferred embodiment hereof, this invention provides a pneumatic medical system, usable in connection with a hand, having fingers and a thumb, comprising, in combination: container means, graspable by the hand, for containing fluid; first passage means and second passage means for defining a path of pressurized fluid through such container means; modulator means for modulating, by use of the thumb, a force of the pressurized fluid through such container means; integral cap means for capping such container means; wherein such integral cap means comprises such modulator means. Moreover, it provides such a pneumatic medical system, further comprising: restraining means for restraining at least one catheter; wherein such integral cap means comprises such restraining means Additionally, it provides such a pneumatic medical system, further comprising: un-clogging means for un-clogging such container means; wherein such integral cap means comprises such un-clogging means.

In accordance with another preferred embodiment hereof, this invention provides a pneumatic medical system, usable in connection with a hand, having fingers and a thumb, comprising, in combination: at least one container, graspable by the hand, adapted to contain fluid; at least one first passage and at least one second passage structured and arranged to define a path of pressurized fluid through such at least one container; at least one modulator adapted to modulate, by use of the thumb, a force of the pressurized fluid through such at least one container; at least one integral cap adapted to cap such at least one container; wherein such at least one integral cap comprises such at least one modulator. Also, it provides such a pneumatic medical system, further comprising: at least one restrainer structured and arranged to restrain at least one catheter; wherein such at least one integral cap comprises such at least one restrainer. In addition, it provides such a pneumatic medical system, further comprising: at least one clip; wherein such at least one restrainer comprises such at least one clip. And, it provides such a pneumatic medical system, further comprising: at least one un-clogger adapted to un-clog such at least one container; wherein such at least one integral cap comprises such at least one un-clogger. Further, it provides such a pneumatic medical system, further comprising: at least one removable replacement lid; wherein such at least one container comprises indicia structured and arranged to assist in determining an amount of substance within such at least one container; and wherein such at least one modulator comprises at least one finger valve.

Even further, it provides such a pneumatic medical system, wherein such at least one modulator comprises at least one finger valve. Moreover, it provides such a pneumatic medical system, further comprising: at least one endotracheal tube adaptor port; wherein such at least one integral cap comprises such at least one endotracheal tube adaptor port. Additionally, it provides such a pneumatic medical system, wherein: such at least one container comprises indicia structured and arranged to assist in determining an amount of substance within such at least one container. Also, it provides such a pneumatic medical system, wherein: such at least one container comprises advertising indicia. In addition, it provides such a pneumatic medical system, further comprising: at least one removable replacement lid. And, it provides such a pneumatic medical system, further comprising: at least one smaller-diameter catheter; at least one larger-diameter catheter structured and arranged to fit into at least one such passage; wherein such at least one smaller-diameter catheter and such at least one larger-diameter catheter are structured and arranged so that such at least one smaller-diameter catheter is connectable to such at least one larger-diameter catheter by being pulled through such at least one larger-diameter catheter. Further, it provides such a pneumatic medical system, wherein such at least one smaller-diameter catheter comprises at least one side hole. Even further, it provides such a pneumatic medical system, wherein such at least one container has a substantially vertical central longitudinal axis. Moreover, it provides such a pneumatic medical system, wherein such at least one container comprises substantially a right circular cylinder.

In accordance with another preferred embodiment hereof, this invention provides a pneumatic medical device, usable in connection with a hand, having fingers and a thumb, for use with at least one container having a substantially vertical central longitudinal axis and structured and arranged to contain a fluid, comprising: at least one integral cap, structured and arranged to cap the at least one container, and comprising: at least one first passage and at least one second passage, structured and arranged, with the at least one container, to define a path of pressurized fluid through the at least one container; and at least one modulator adapted to modulate, by use of the fingers or thumb, a force of the pressurized fluid through such at least one container. Additionally, it provides such a pneumatic medical device, wherein such at least one integral cap further comprises at least one restrainer structured and arranged to restrain at least one catheter. Also, it provides such a pneumatic medical device, wherein such at least one modulator comprises at least one finger valve. In addition, it provides such a pneumatic medical device, wherein such at least one integral cap further comprises at least one endotracheal tube adaptor port. And, it provides such a pneumatic medical device, further comprising: at least one clip; wherein such at least one restrainer comprises such at least one clip. Further, it provides such a pneumatic medical device, wherein such at least one integral cap further comprises at least one un-clogger adapted to un-clog the container.

Even further, it provides such a pneumatic medical device, wherein: at least one such passage comprises at least one downwardly-extending passage extension; and at least one such passage comprises such at least one modulator. Moreover, it provides such a pneumatic medical device, further comprising: at least one smaller-diameter catheter; at least one larger-diameter catheter structured and arranged to fit into at least one such passage; wherein such at least one smaller-diameter catheter and such at least one larger-diameter catheter are structured and arranged so that such at least one smaller-diameter catheter is connectable to such at least one larger-diameter catheter by being pulled through such at least one larger-diameter catheter. Additionally, it provides such a pneumatic medical device, wherein such at least one smaller-diameter catheter comprises at least one side hole. Also, it provides such a pneumatic medical device, wherein the longitudinal axes of such first passage, such second passage, and the container, are essentially parallel. In addition, it provides such a pneumatic medical device, further comprising: at least one connector structured and arranged to connect at least one of at least one first passage and at least one second passage to at least one pressurized fluid source. And, it provides such a pneumatic medical device, wherein; at least one adaptor structured and arranged to attach at least one of such at least one first passage and at least one second passage to at least one fluid splash shield. Further, it provides such a pneumatic medical device, wherein such at least one adaptor comprises at least one luer adaptor.

In accordance with another preferred embodiment hereof, this invention provides a pneumatic medical system comprising: at least one smaller-diameter catheter; and at least one larger-diameter catheter; wherein at least one of such at least one smaller-diameter catheter and such at least one larger-diameter catheter comprises at least one flexible catheter; wherein such at least one smaller-diameter catheter is releasably connected, via an airtight frictional fit, within such at least one larger-diameter catheter; wherein such at least one smaller-diameter catheter and such at least one larger-diameter catheter each comprise a proximal and a distal end; and wherein such distal end of such at least one smaller-diameter catheter extends beyond such distal end of such at least one larger-diameter catheter.

In accordance with another preferred embodiment hereof, this invention provides a pneumatic medical system comprising, in combination: at least one source of oxygen under pressure; at least one container adapted to contain irrigation fluid; wherein such at least one container comprises at least one inlet adapted to inlet such oxygen under pressure from such at least one source into an interior upper portion of such at least one container; and wherein such at least one container comprises at least one outlet adapted to outlet such irrigation fluid from an interior lower portion of such at least one container; and at least one connector adapted to connect such at least one source with such at least one container in such manner as to assist forming a passageway for such oxygen under pressure from such at least one source to enter such at least one inlet; wherein, when such at least one container contains such irrigation fluid and when such oxygen under pressure enters such at least one container, such irrigation fluid will expel through such at least one outlet and thus be available for irrigation use. Even further, it provides such a pneumatic medical system further comprising a splash shield system having a fluid connection with such at least one outlet.

In accordance with another preferred embodiment hereof, this invention provides a pneumatic medical system comprising the steps of: providing a sealed container of irrigation fluid, such container comprising at least one unsealable inlet and at least one unsealable outlet; unsealing such container by unsealing such at least one unsealable inlet and such at least one unsealable outlet; connecting such unsealed at least one unsealable inlet with a source of oxygen under pressure; and using the irrigation fluid expelled from such container when oxygen enters such unsealed at least one unsealable inlet for irrigation.

### BRIEF DESCRIPTION OF THE DRAWINGS

FIG. 1 is a perspective view of the prior art DeLee catheter and trap.

FIG. 2 is a perspective view of the prior art DeLee trap tilted on its side.

FIG. 3 is a perspective view of a preferred embodiment of the pneumatic medical system of the present invention.

FIG. 4 is a rear sectional view through the preferred embodiment of FIG. 3.

FIG. 5 is a top view of the preferred pneumatic medical system of FIG. 4.

FIG. 6 is an expanded partial perspective view of the preferred embodiment of FIGS. 3-5, illustrating a preferred catheter clip embodiment of the present invention.

FIG. 7 is a perspective view of the pneumatic medical system of FIGS. 3-6, particularly illustrating how such system may be held and operated with one hand.

FIG. 8 is a sectional view of another preferred embodiment of the pneumatic medial system of the present invention illustrating the use of such embodiment to force fluid flow.

FIG. 9 shows another preferred embodiment for tissue irrigation using an external fluid source.

FIG. 10 is a perspective view showing another preferred embodiment of the present invention.

FIG. 11 is a sectional view through section 11-11 of FIG. 10 showing, in detail, the various interior assemblies of the pneumatic medical system of the present invention.

FIG. 12 is a perspective view showing another preferred embodiment of the pneumatic medical system of the present invention.

FIG. 13 is a sectional view through section 13-13 of FIG. 12 showing, in detail, the various interior assemblies of the pneumatic medical system of the present invention.

FIG. 14 is a perspective view of a disposable pre-filled irrigator according to a preferred embodiment of the present invention.

FIG. 15 is a perspective view, partially in section of the upper portion of the disposable pre-filled irrigator of FIG. 14.

FIG. 16 is a sectional view through section 16-16 of FIG. 14.

FIG. 17 is a sectional view of the upper portion of the disposable pre-filled irrigator illustrated in FIG. 16 illustrating another preferred embodiment of the disposable pre-filled irrigator.

FIG. 18 is a perspective view of an irrigator or suction device according to a preferred embodiment of the present invention.

### DETAILED DESCRIPTION OF THE PREFERRED EMBODIMENTS AND BEST MODES OF PRACTICE

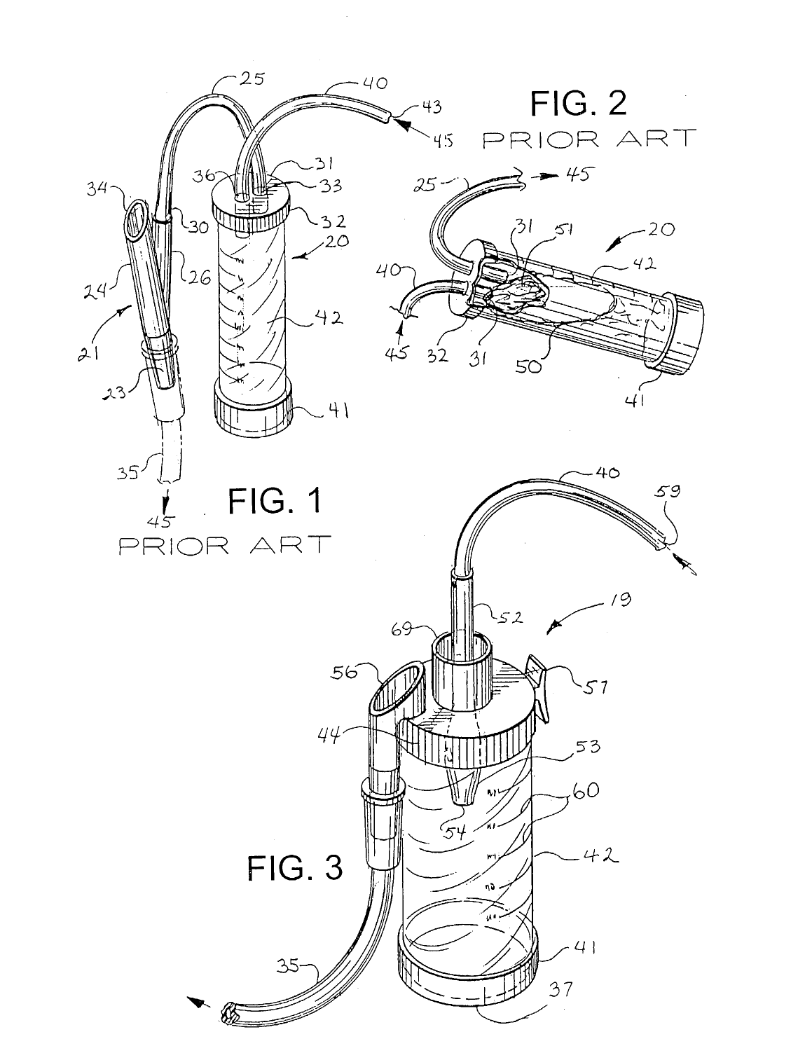

FIG. 1 illustrates the prior art. Shown in FIG. 1 is a specimen trap, also called a DeLee trap or DeLee trap system 20. Illustrated in FIG. 1 is outlet catheter 35. Outlet catheter 35 is attached at its distal end to a well-known vacuum source, such as a wall-mounted adapter, well known in the art (not shown). Outlet catheter 35 is attached at its proximal end to the cylindrical hollow bottom end 23 of the Y-shaped thumb valve 21. At the opposite end from bottom end 23 of Y-shaped thumb valve 21 are two cylindrical hollow tubes, a valve tube 24 and a catheter tube 26. Valve tube 24 and catheter tube 26 fuse to form cylindrical hollow bottom end 23 (as shown). Outlet catheter 25 has a distal connection end 30 and a proximal canister cap end 31 (as shown). Connection end 30 of outlet catheter 25 is connected to catheter tube 26. Canister cap end 31 of outlet catheter 25 passes through canister cap 32 through hole 33. Valve tube 24 has a diagonal top end 34. Also attached to canister cap 32, here through hole 36, is inflow catheter 40 (as shown).

When outlet catheter 35 is attached to a suction source (not shown) and the suction is turned on, air or fluid is pulled through the openings in the system 20. For example, in the open position, air is pulled through the open diagonal top end 34 of Y-shaped thumb valve 21, through valve tube 24 and through outlet catheter 35 to the source of suction. When diagonal top end 34 of Y-shaped thumb valve 21 is occluded, by placing a thumb firmly over diagonal top end 34 of Y-shaped thumb valve 21, the system 20 is closed except for the opening at the aspiration end 43 of inflow catheter 40. Therefore, when diagonal top end 34 of Y-shaped thumb valve 21 is occluded, air is pulled through inflow catheter 40, through canister 42, through outlet catheter 25, through catheter tube 26 of Y-shaped thumb valve 21, through bottom end 23 of Y-shaped thumb valve 21, and through outlet catheter 35 to the suction source. The direction of flow through the system is illustrated by arrows 45.

When suction is applied, and the diagonal top end 34 of Y-shaped thumb valve 21 is occluded, and the aspiration end 43 of inflow catheter 40 is placed in liquid (and/or other material to be suctioned), some of such liquid is pulled through the inflow catheter 40 and flows into canister 42. Such liquid falls to the bottom of canister 42, by virtue of gravity. Displaced air, or air sucked through inflow catheter 40 along with aspirated liquid, will then flow through outlet catheter 25, through catheter tube 26 of the Y-shaped thumb valve 21, through bottom end 23 of Y-shaped thumb valve 21, and out through outlet catheter 35 to the suction source.

After suctioning is complete, and the suctioned liquid has been collected in the canister 42, the canister cap 32 can be removed and replaced with the replacement lid 41 that is packaged attached to the bottom of canister 42. Replacement lid 41 does not have holes 33 or 36. Replacement lid 41 can form a tight seal with canister 42, allowing canister 42 to be transported to a laboratory for analysis of its enclosed material with a reduced risk of spilling, leaking, or falling over.

FIG. 2 illustrates one significant problem with this prior art DeLee trap system 20. FIG. 2 illustrates the canister 42 tipped at an angle. If, during a suctioning procedure, the canister 42 is allowed to tip over at an angle, as illustrated in FIG. 2, liquid 50 or small solid samples 51 can occlude the canister cap ends 31 of outlet catheter 25 and inflow catheter 40. If canister cap ends 31 are blocked, suction will not flow past the blockage, rendering the system inoperable. Once blocked, these systems may be very difficult to clear. Another significant problem with this prior art device/system 20 is that it is awkward to use. As discussed, the canister 42 must be held upright, or the system might clog. This requires that the user hold the canister 42. The user must also hold and operate Y-shaped thumb valve 21. In addition, the user must manipulate the inflow catheter 40 that is often long and flimsy. The inflow catheter 40 may be introduced into an infant's nose or mouth to remove liquid or meconium. In addition, the suction must be turned on at the source, often on a wall behind the patient's bed. Turning on the suction source also requires a hand. And, the operator might be actively involved in supporting the head of a newborn during and immediately after birth. One hand must maintain the connection of the bottom of the thumb valve 23 to the outlet catheter 35, while also controlling the thumb valve opening 34, while also gripping and orienting the canister 42, while the operators second hand manipulates the flexible catheter 25. Simultaneously, the user may also need to stabilize the patient, for example, controlling a newborn baby's head during a delivery using a combination of hands, arms and other body parts.

It would be desirable, in these cases, to have a device with a specimen trap, such as a DeLee, that is easier to handle. This device might have fewer parts and might be in a more ergonomic relationship. Ideally, this device would be more inexpensive to manufacture. For example, a single part might replace multiple parts, requiring multi-stepped assembly. The single part might be injection-molded. An ideal design for a part might be one that can be manufactured in a linear arrangement to eliminate the need for more costly side actions on the mold. It would, therefore, be desirable to have a molded part that could fit over a trap canister, such as that used with a DeLee trap, that could eliminate the need for assembly of parts that would be designed with the inlet and outlets in a collinear relationship to facilitate even more economical manufacturing. DeLee traps are sometimes used for tracheal suctioning, particularly, to clear meconium aspiration. DeLee traps usually use an 8Fr or 10Fr catheter for nasogastric suctioning of a full-term newborn. Some procedures call for larger catheters, 12Fr or 14-16Fr. Changing catheters can be time-consuming and distracting. It would be desirable to have a system of catheters arranged to have both small and large catheters immediately available, using the same device with a minimum of manipulations. There are devices that have tips of varying diameters to control flow, but the outer diameters remain fixed. They, therefore, do not permit a single device with a large outer diameter tubing to be used on a patient with small diameter tubing needs, and then to be immediately changed to large diameter tubing, if the need arises.

It would be desirable to have such a system that could operate using a minimal amount of parts or tooling required to create the necessary parts. Frequently different sized tubing is connected, via molded connectors, such as luer locks. These must be molded, and then bonded or welded to the tubing. This process can be labor-intensive and requires great care for quality control, and therefore, makes products more expensive to produce. It would be desirable to have a single device that might accommodate different user preference by having multiply-dimensioned tubing attached to the device that would be operable and could be immediately changed to another operable form. Having such a device would save time by eliminating the need to locate the desired secondary devices, and it would simplify stocking, as one device would fulfill the function of at least two. In some cases, it might be desirable to have such a connection irreversible once broken for reasons including safety or recognition.

Another medical setting in which a pneumatic system could be used is when it is sometimes necessary to administer fluids, such as sterile fluids or intravenous (IV) fluids commonly available in the hospital, including saline, rapidly to a patient. Often, IV fluids are delivered into the patient-by means of an IV bag holding the medication or fluid to be delivered to the patient above the patient. The IV bag is connected to a catheter that is attached to a needle directed toward the patient. The fluids flow toward the patient by means of gravity from the bag suspended above the patient. Sometimes it is desirable or necessary to deliver those fluids rapidly toward the patient. For example, if the patient has suffered traumatic injury and has lost significant amounts of blood, the rapid delivery of replacement fluids can be a life-saving procedure, or may help with immediate cleansing of wounds. Usually, if there is a need to deliver fluids to the patient rapidly, a medical professional is required to squeeze the IV bag, or a rapid infuser is used to mechanically apply pressure to the IV bag. This procedure of squeezing a bag, which may be suspended above the level of the medical professional's head, can be extremely tiring and difficult. Rapid infusers can be very expensive.

In addition, it is sometimes necessary to administer fluids, such as saline, rapidly or under high pressure, to a patient, including situations such as wound irrigation, where it is desirable to use large amounts of sterile fluid under high pressure. Currently, techniques involve the use of a syringe, which is limited by the volume. Typically, syringes are no larger than 20-60 cc. To administer larger amounts of sterile solution, the medical personnel must draw up fluid from a reservoir, discharge and then refill the syringe multiple times, which is labor-intensive and detrimental in a busy emergency room.

Morse describes administering fluid by manually squeezing a bottle or bag with a syringe tip. The reservoir is larger, but when near empty, the gas-to-liquid ratio is greater, and is more difficult to generate enough pressure by compressing a bottle for wound irrigation. Other difficulties with this arrangement are that the pressures are not regulated, nor even continuous. This makes creating protocols more difficult. The manual pumping requirement is tiresome and is a disincentive for optimal wound care using the currently available techniques. More costly electronic pumps are available, but their complexity and cost is prohibitive for universal and disposable use, and is, therefore, used in only a few select situations.

Frequently, the fluid source is a reservoir, such as a bottle or canister, or bag of fluid. The prior art does not describe a pressurized inlet fluid source, connected to an external pressurized fluid source, and pressurized to expel a sterile liquid from a disposable liquid reservoir for medical purposes, including wound irrigation.

Canisters are commonly used in pneumatic medical systems. The current canister arrangements are adapted for suctioning purposes only to prevent clogging of suction traps. They have both inlet and outlet ports in upper positions to prevent clogging when debris is captured. If one wanted to expel a liquid from such a closed canister, bottle or other reservoir with the use of an externally pressurized fluid, the outlet port of the reservoir would need to be at or near the bottom of the bottle where the liquid would occlude the outlet port, preferably, without flowing out uncontrollably. This is not present in the prior art; nor is it described in a simple and inexpensive, disposable arrangement. When a pressurized fluid is applied to a closed system, the liquid obstructing the outflow channel would be preferentially expelled to release pressure in the closed system. Currently available equipment does not have conduits and adaptors to mate with commonly available pressurized fluid sources, or regulated pressurized fluid sources in medical settings, such as hospitals. They do not have adaptors to connect with oxygen sources, such as oxygen tanks or oxygen regulators connected to oxygen tanks or wall sources.

These systems do not have a means to vary the diameter of the outflow conduit to vary the pressure of the fluid effluent; nor are they adapted on the outlet to mate with a wound irrigation shield to contain the pressurized effluent; nor is there taught a means to integrate or attach such a pressurized system with commonly available large fluid reservoirs, such as a bottle of sterile fluid, including saline, by using a lid, such as a specially adapted cap or plug; nor is there taught in the prior art a multipurpose system which can be modified from a pneumatic pressurized irrigation system to a pneumatic suction system by distraction and repositioning of an outflow tube, enabling a single device to serve both purposes. Having such a multipurpose device might allow an irrigation bottle to be emptied using a pressurized oxygen system, for example, and then the device could be attached to a vacuum source. An inner telescopically connected catheter attached to a wound irrigation shield could be removed, and then the device could then suction the contaminated fluid back into the irrigation bottle for self-contained disposal. It would be desirable to have such a system, as it would reduce linen costs, and would reduce the need for larger and more costly suction canister units; as in this example, the emptied irrigation bottle would have been useless disposed trash; whereas, it is now useful, and could be immediately “recycled”.

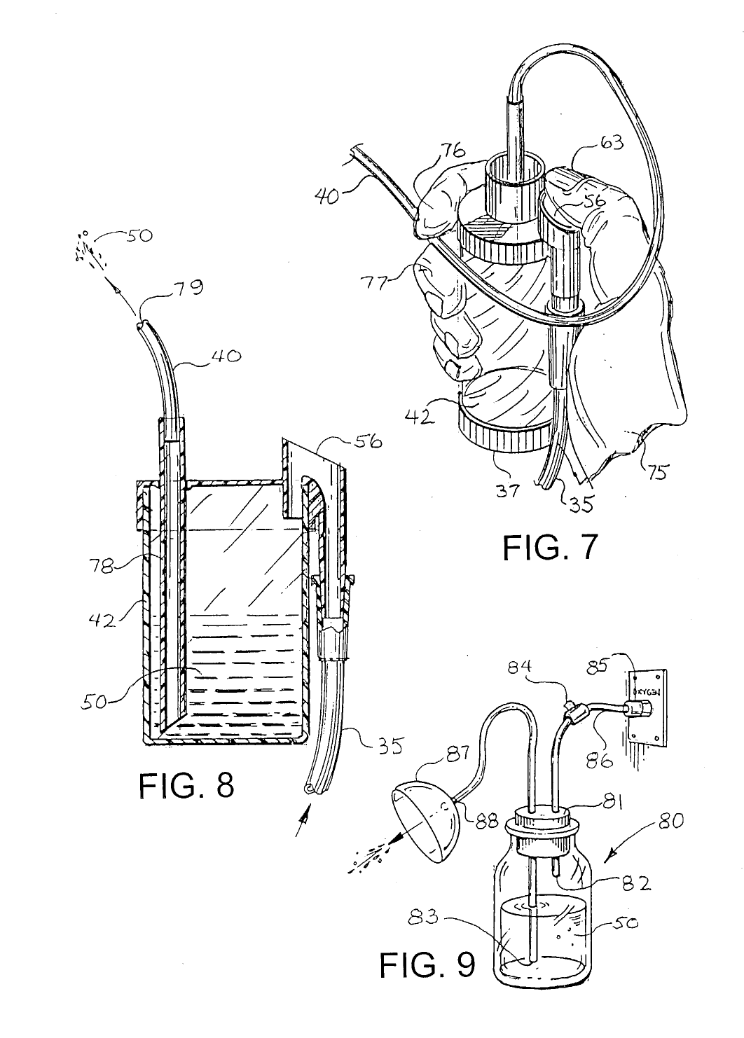

FIG. 3 illustrates a preferred embodiment of the pneumatic medical system 19 of the present invention. FIG. 3 illustrates that the integral canister cap 44 (herein embodying integral cap means) preferably includes an integral thumb valve 56 (herein embodying modulator means for modulating a force of the pressurized fluid through such container means wherein such modulator means comprises at least one passage) and catheter restraint 57 (herein embodying restraining means for restraining at least one catheter), as shown. Incorporating these features into a single integral canister cap 44 makes handling of this device more ergonomic and less awkward to use than prior art devices, particularly when single-hand manipulation is required. System 19 (herein embodying such a system wherein the longitudinal axes of such first passage, such second passage, and the container are essentially parallel) eliminates the need for a second outlet catheter (See catheters 25 and 40, both emerging from canister cap 32 as illustrated in FIGS. 1 and 2). By preferably integrating the thumb valve 56 into the integral canister cap 44 (herein embodying at least one integral cap), the operator is not required to hold onto two separate elements, the canister 42 and a separate thumb valve such as the Y-shaped thumb valve 21 as illustrated in FIG. 1. Using this device/system 19, the integral thumb valve 56 (herein embodying at least one modulator wherein such at least one modulator comprises at least one finger valve) may be operated using the same hand that is holding the canister 42 (herein embodying container means and herein embodying at least one container structured and arranged to contain a fluid wherein such at least one container has a substantially vertical central longitudinal axis and wherein such at least one container comprises substantially a right circular cylinder, See FIG. 7 for an example). Preferably, the outlet adaptor 61 (herein embodying at least one connector structured and arranged to connect at least one such passage to at least one pressurized fluid source) is integral with the modulator valve 56 and with the cap 44. This allows the palm of a single gripping hand to maintain the connection with the outlet with the outlet hose 35, while simultaneously gripping the canister 42, modulating the flow valve and directing the catheter 40. Catheter restraint 57 (herein embodying at least one restrainer and herein embodying at least one clip) may preferably be used to hold inflow catheter 40 in place and out of the way when desired and may also be used to “clip” the entire canister system 19 to a nearby piece of equipment, as shown.

FIG. 3 illustrates inflow catheter 40 (herein embodying at least one smaller-diameter catheter) preferably passing through integral canister cap 44 through telescoping catheter 52 (herein embodying at least one larger-diameter catheter). Preferably, inflow catheter 40 is made of flexible (preferably approximately 70-85 durometer), radiopaque, approximately 8 Fr (8 French) catheter tubing material and may usefully be approximately 14 inches in length. Preferably, the telescoping catheter 52 is of larger diameter, approximately 14Fr (14 French) and stiffer (preferably 85-95 durometer) material. Preferably, the proximal end of the inflow catheter 40 is slightly flared to assist in forming a telescopic fit within the larger proximal telescoping catheter 52. Preferably, this slight flare in the inflow catheter 40, inside the slight (bulbous) enlargement 53 of the telescoping catheter 52, will allow these two tubes to hold together with a friction fit, without constricting the diameter of either catheter. Preferably, both the inflow catheter 40 and the telescoping catheter 52 are made of PVC material that tends to adhere to itself (under appropriate circumstances other medical grade plastic materials, such as silicone, may suffice).

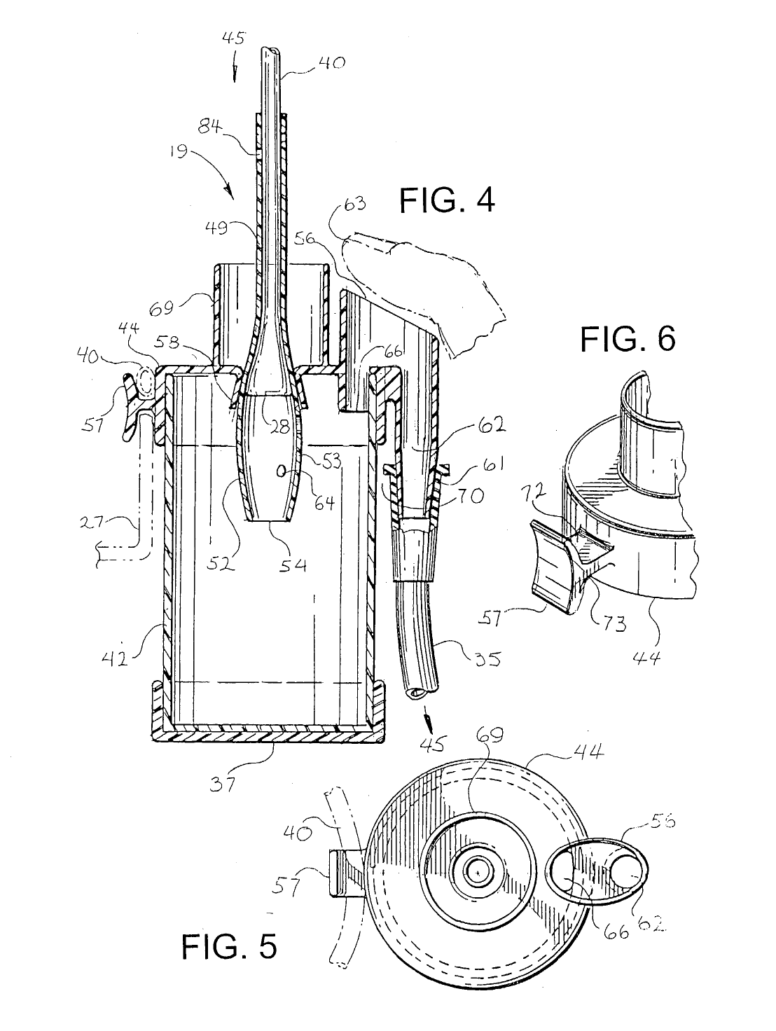

Preferably, when the inflow catheter 40 is pulled through telescoping catheter 52 until the flared end 28 of the inflow catheter 40 lodges in place inside telescoping catheter 52 (herein embodying such at least one smaller-diameter catheter and such at least one larger-diameter catheter structured and arranged so that such at least one smaller-diameter catheter is connectable to such at least one larger-diameter catheter by being pulled through such at least one larger-diameter catheter), the PVC material will form a watertight and airtight coupling internally without additional parts such as Leur-Lock adapters. FIG. 3 also illustrates that telescoping catheter 52 may preferably extend into canister 42. Preferably, telescoping catheter 52 is a catheter with a dual tapered fitting or enlargement 53 on its proximal end 54 (also illustrated in FIG. 4). Telescoping catheter 52 may preferably be tapered so that proximal end 54 of telescoping catheter 52 can fit into a standard suction coupling. Telescoping catheter 52 can preferably be engaged inside catheter holder 58 (as shown in FIG. 4) by pulling upward on telescoping catheter 52 until it achieves a tight fit against catheter holder 58 (herein embodying passage means and herein embodying at least one such passage). Engaged in this way, telescoping catheter 52 achieves an airtight and watertight fit against catheter holder 58, which is integral with integral canister cap 44 (also illustrated in FIG. 4).

FIG. 3 also illustrates that the canister 42 can be marked to measure volume. Markings 60 (herein embodying indicia structured and arranged to assist in determining an amount of substance within such at least one container) may preferably show the volume of liquid contained in the canister 42. Canister 42 may also preferably be marked with advertising indicia (not shown). FIG. 3 also illustrates that a replacement lid 39 (herein embodying at least one removable replacement lid) may be removably attached to the bottom of the canister 42. Once a specimen is collected, the integral canister cap 44 can be removed and replaced with the replacement lid 39. The canister 42, containing a specimen, with the replacement lid 39 attached, can preferably be transported to a laboratory for analysis with reduced chances of spilling or leaking.

FIG. 4 is a sectional view further illustrating this preferred embodiment of the pneumatic medical system of FIG. 3. In this preferred embodiment, outlet catheter 35 is attached at its distal end to a vacuum source (not shown). Outlet catheter 35 attaches at its proximal end to the integral adapter 61 that is integrally attached to integral thumb valve 56, as shown. Integral adapter 61 is preferably cylindrical and defines an internal hollow space 62 through which air flows, as shown. At the top of integral adapter 61 is integral thumb valve 56. As illustrated in FIG. 4, if a thumb 63 is not pressed against integral thumb valve 56, the integral thumb valve 56 provides an opening in the system and air is pulled through the open integral thumb valve 56, through internal hollow space 62, through the outlet catheter 35, to the vacuum source (not shown). Preferably, if thumb 63 is pressed against integral thumb valve 56, the system is closed except for the opening at the aspiration end 59 of the inflow catheter 40. Preferably, when the thumb valve 56 is closed, air is pulled through the aspiration end 59 of the inflow catheter 40, through telescoping catheter 52, into canister 42, through the vacuum port 66, past the closed integral thumb valve 56 and out through outlet catheter 35. Preferably, when a thumb 63 is pressed against integral thumb valve 56, and the aspiration end 59 of inflow catheter 40 is placed in liquid (and/or other material), such liquid/material will preferably flow through the inflow catheter 40, through telescoping catheter 52, and flow into canister 42. Such liquid/material will fall to the bottom of canister 42 by virtue of gravity. Displaced air, or air sucked through inflow catheter 40 along with liquid, will then flow through outlet catheter 35 to the vacuum source. Arrows 45 indicates airflow through the device.

Telescoping catheter 52 is preferably 14Fr (14 French) semi-rigid tubing, slightly larger and more rigid than the preferable 8Fr (8 French) tubing for the inflow catheter 40. If a larger diameter catheter is desired for suction, inflow catheter 40 can preferably be removed from the device by simply pulling it out of its connection with telescoping catheter 52. In this way, telescoping catheter 52, which is of a larger diameter and stiffer, can preferably be used directly for suctioning. Telescoping catheter 52 preferably has a hole 64 (herein embodying at least one side hole). Hole 64 preferably functions to increase the capacity of canister 42. In the absence of hole 64, if canister 42 is filled with liquid that reaches the level of the bottom end 54 of the telescoping catheter 52, the system could become clogged. Hole 64 preferably functions as a safety feature to permit the outflow of fluid should the bottom end 54 of the telescoping catheter 52 become clogged. Hole 64 also functions preferably to allow a higher level of fluid to be attained inside the canister 42 without the device losing its function. In addition, should canister 42 become filled with fluid (and so clogging it), this apparatus can preferably be tipped (convenient with the hand holding it) to allow liquid to flow out through vacuum port 66 and through integral thumb valve 56. Or, the apparatus can be tipped, and the thumb 63 remain engaged on the integral thumb valve 56, to allow trapped fluid to flow through the outlet catheter 35 to the vacuum source (these described arrangements embodying un-clogging means according to this invention). Telescoping catheter 52 and inflow catheter 40, telescopically attached to each other, can preferably be removed from the integral canister cap 44 by pulling the thinner length of telescoping catheter 52, or by removing integral canister cap 44 from canister 42 and pulling the thinner length 49 of telescoping catheter 52, containing the proximal end of inflow catheter 40 through catheter holder 58. If suction without specimen collection is required, the telescoping catheter 52 can preferably be connected to a readily available suction catheter adapter at its bottom end 54. Therefore, a well-known vacuum source can preferably be attached to a well-known suctioning catheter with a well-known adapter and the suctioning catheter adapter can be attached to the bottom end 54 (proximal end) of telescoping catheter 52. Hole 64 can preferably be used to regulate the level of suction by placing a finger over hole 64 or removing the finger (a finger flow control valve). If hole 64 is occluded by a finger, air can preferably be pulled through the inflow catheter 40, through telescoping catheter 52, through a suction catheter adapter, through a suction catheter to the vacuum source. If hole 64 is left open, air can preferably be pulled through hole 64, through bottom end 54 (proximal end) of telescoping catheter 52, through a suction catheter adapter, through a suction catheter to the vacuum source. If large volume suctioning, or suctioning in a large orifice is required, larger diameter telescoping catheter 52 can preferably be used alone (and smaller diameter inflow catheter 40 can be removed by pulling it through telescoping catheter 52). If smaller volume suctioning, or suctioning in a smaller orifice is required, smaller diameter inflow catheter 40 can be left attached to telescoping catheter 52. If inflow catheter 40 is removed from telescoping catheter 52 and hole 64 is occluded by a finger, air will preferably be pulled through telescoping catheter 52, past hole 64, through a suction catheter adapter, through a suction catheter to the vacuum source. Hole 64 can also preferably be left unoccluded by a finger, allowing air to be pulled through hole 64, through the bottom end (proximal end) of telescoping catheter 52, through a suction catheter adapter, through a suction catheter to the vacuum source, in the same manner as described above. Attaching the telescoping catheters in this manner reduces the requirement for connecting parts such as Leur Locks and other types of adaptors. These parts require expensive tooling to manufacture and expensive assembly to attach them to catheter tubing. Usually, the distal tips of suction catheters have at least one safety eyelet 38 or aperture punched in the sidewall of the catheter. This feature protects delicate tissues from being suctioned too forcefully. In this preferred embodiment, when the smaller diameter inflow catheter 40 is pulled through the larger diameter telescoping catheter 52, the proximal end of inflow catheter 40 is lodged inside the distal end of telescoping catheter 52. When secured together in this manner (herein embodying such at least one smaller-diameter catheter releasably connected via an airtight frictional fit within such at least one larger-diameter catheter; wherein such at least one smaller-diameter catheter and such at least one larger-diameter catheter each comprise a proximal and a distal end; and wherein such distal end of such at least one smaller-diameter catheter extends beyond such distal end of such at least one larger-diameter catheter), the proximal end of inflow catheter 40 seals over any safety eyelet or aperture which might be present in the distal end of the more proximal telescoping catheter 52. This feature preferably prevents loss of vacuum power when the two catheters, telescoping catheter 52 and inflow catheter 40 are used together. And, if it is preferred to only use the larger-diameter telescoping catheter 52, once the inflow catheter 40 is removed, safety eyelets or apertures may be present on the distal end of the telescoping catheter 52 for safe suctioning using that catheter alone. FIG. 4 also illustrates adaptor port 69 (herein embodying at least one endotracheal tube adaptor port). Adaptor port 69 could preferably be used to clear meconium from a newborn baby's lungs. Inflow catheter 40 and telescoping catheter 52, could preferably be removed from the canister 42, as described above, and used independently of the canister 42, as described above, to suction the patient's oropharynx (without collecting any liquid or solid specimens in canister 42). Then the patient could be separately intubated. Preferably, then, the outlet catheter 35 could be attached to a vacuum source, the operator could place a thumb 63 over the integral thumb valve 56, creating a vacuum through the only remaining opening in the system, catheter holder 58 which opens into adaptor port 69. Then, adaptor port 69 could be briefly attached to a typical endotracheal tube adaptor to assist in clearing meconium from the patient's lungs after intubation. Adaptor port 69 preferably would have a dimension of approximately 15 mm diameter to enable attachment of the device over such typical endotracheal tube adaptor.

FIG. 4 also illustrates a recess 70 between the integral adapter 61 attached to outlet catheter 35, and canister 42. This recess 70 can preferably be used to hang the device from another surface. For example, the device can be assembled and hung from the side of a bed during delivery. Then when the device is required to suction the newborn, it would be within easy reach and easily available to the physician or midwife when needed.

FIG. 5 is a top view illustrating the integral canister cap 44. The integral thumb valve 56 is illustrated. Also illustrated is the internal hollow space 62 defined by the cylindrical integral adapter 61. Also shown is vacuum port 66. This top view illustrates that vacuum port 66 forms a continuous passageway between the top opening of the integral thumb valve 56 and the inside of the canister 42. Preferably, if the level of liquid is too high inside canister 42, the device can be tipped so that liquid flows from canister 42 through vacuum port 66 and out through integral thumb valve 56. Also illustrated from the top is the adaptor port 69 for connecting to an endotracheal tube adaptor. And, illustrated is a loop of catheter, preferably inflow catheter 40, clipped in catheter restraint 57.

FIG. 6 is an expanded partial perspective view of the system 19, illustrating catheter restraint 57 integrally attached to integral canister cap 44. Preferably, catheter restraint 57 is two-sided so that a loop of catheter, preferably inflow catheter 40, can be clipped into the top 72 of the catheter restraint 57 or into the bottom 73 of catheter restraint 57. In addition, catheter restraint 57 may preferably hold two loops of catheter at the same time. The bottom location 73 of restraint 57 may also act as a “clip” on other equipment 27 to hold the device of system 19 in a convenient location when not in use (see the dotted line in FIG. 4 showing possible such equipment 27).

FIG. 7 illustrates the use of the device/system 19 as it is held by a hand 75. The thumb 63 is shown in position to occlude the integral thumb valve 56. This embodiment shows the inflow catheter 40 held between the forefinger 76 and middle finger 77 of the hand 75. FIG. 7 illustrates that the device 19 preferably fits comfortably in a hand and may be held and operated with a single hand. FIG. 7 also illustrates that, since the whole apparatus (of system 19) is being held by the operator, the orientation of the canister 42 can be better controlled. Preferably, if solid material is inside the canister 42, the operator can avoid clogging by solid material by controlling the orientation of the canister 42 (herein embodying un-clogging means for un-clogging such container means and herein embodying at least one un-clogger) and preventing the type of clogging illustrated in FIG. 2. Because the operator is holding this canister 42 while operating this device, controlling the orientation of the canister may not be awkward.

FIG. 8 illustrates that the device can also be used to force liquid 50, such as saline, out of a canister 42. This preferably makes the system a multi-functional device that can be used alternatively for irrigation or for specimen collection by repositioning of the proximal end of the telescoping catheter 52. In place of a vacuum source, the outlet catheter 35 can be attached to a pressurized fluid (wherein said pressurized fluid source may preferably comprise: a portable oxygen tank; a hospital gas supply unit; a hospital oxygen supply unit; a wall fluid supply unit; and a wall oxygen supply unit). Oxygen can preferably flow into outlet catheter 35. If the integral thumb valve 56 is unoccluded, that oxygen would preferably flow straight out through the integral thumb valve 56. If a thumb 63 (not shown) is placed over the integral thumb valve 56, the oxygen would preferably flow into the canister. As illustrated in FIG. 8, if the canister is filled with liquid 50 and the only other opening in the system is the distal end 79 of the inflow catheter 40, the pressure from the pressurized fluid would cause liquid 50 to flow up through the modified telescoping catheter 78 (shown here without a hole 64) and out through the inflow catheter 40. The flow of liquid could be modulated by partially or wholly covering the integral thumb valve 56 with a thumb 63. Flow through this system is illustrated by arrows 45. Flow may also be modulated by a fluid regulator (not shown) intermediate to the fluid source.

FIG. 9 shows another preferred embodiment with a lidded container 80 with lid 81 containing liquid 50 and having a pressure inlet 82 and pressure outlet 83 located at the gravitational base of the container 80. Also shown is a finger flow control valve 84 intermediate the regulated positively pressurized fluid source 85, shown herein as an oxygen tank. The connector 86 is a gas-tubing connector of standard dimensions that connects the inlet fluid source via a conduit to the finger flow control valve. Also illustrated is an optional splash shield 87 connected via a standard medical connector 88 (herein embodying at least one adaptor structured and arranged to attach at least one such passage at least one fluid splash shield), such as luer fitting, to contain the pressurized fluid 50 when used for purposes such as wound irrigation.

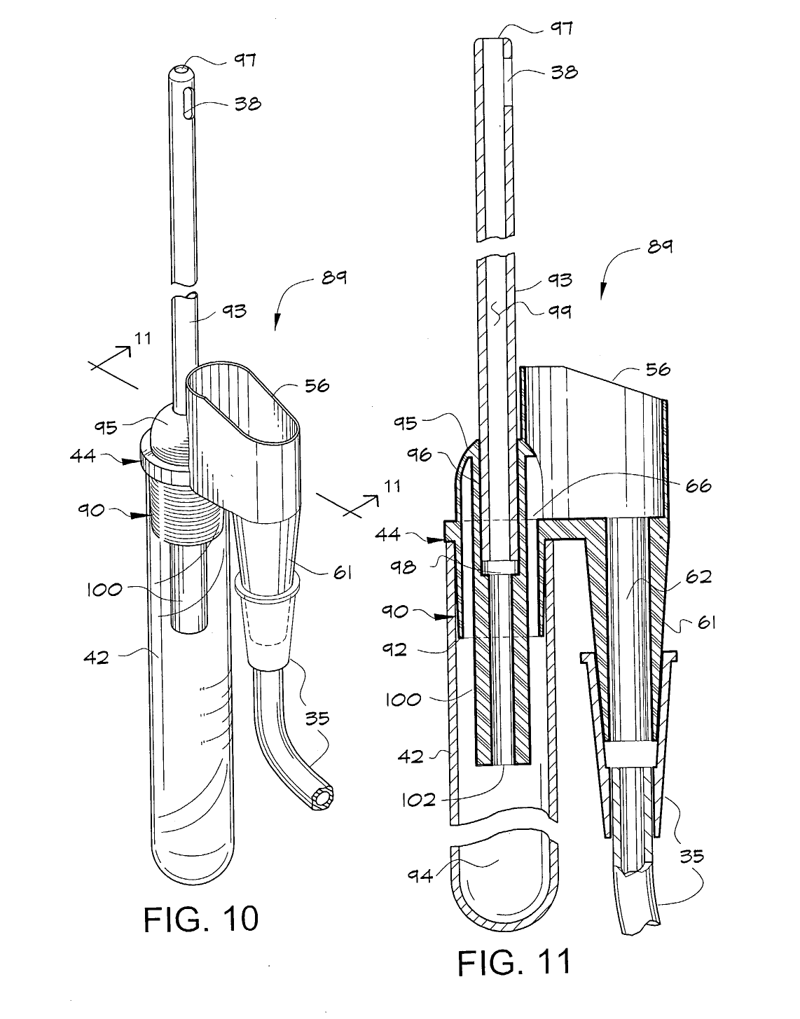

FIG. 10 is a perspective view showing another preferred embodiment of the present invention. In the pneumatic medical system of FIG. 10, a simplified integral cap assembly, having a fixed catheter 93 (herein embodying passage means) and integral catheter connector 95, is used (in lieu of the telescoping catheter assembly described previously). Although somewhat less adaptable, the use of a fixed catheter system 89 holds a number of advantages over the telescoping catheter system including ease of manufacture and assembly combined with reduced cost. An additional advantage of the fixed catheter system 89 is that the integral canister cap 44 may be compactly designed to allow for use with small diameter canisters (e.g. small caliber culture tubes), as shown. Preferably, the fixed catheter system 89 comprises at least one fixed catheter 93 inserted into a catheter connector 95, as shown. Preferably, the fixed catheter 93 is a rigid suction type, of uniform diameter, comprising approximately 14Fr (14 French) and stiffer (preferably 85-95 durometer) material (under appropriate circumstances a more flexible catheter may be utilized). Preferably, the fixed catheter 93 is permanently bonded to the catheter connector 95 however, under appropriate circumstances, the fixed catheter 93 may be removably joined to the catheter connecter 95 by friction fit or by other standard medical connector means. By preferably allowing the removal of the fixed catheter 93, the operator of the device may switch integral canister cap 44(s) and canister 42(s) as needed, without removing the suction tip 97 (of the fixed catheter 93) from the target suction point. In both the removable and bonded catheter embodiments, the catheter connector 95 is structured to firmly secure the fixed catheter 93 in a manner that will not cause a constriction of the inner lumen of the tubing 99 with the insertion process.

Preferably, the catheter connector 95 is integrally formed to the upper portion of the integral canister cap 44, located such that the fixed catheter 93, catheter connector 95 and canister 42 share the same approximate longitudinal central axis, as shown. In the embodiment of FIG. 10, the lower portion of the integral canister cap 44 is preferably provided with at least one integral stopper 90, comprising at least one annular shaped tapered segment 92 adapted to snuggly fit within the upper interior of the canister 94, as shown. Engaged in this way, the integral canister cap 44 achieves an airtight and watertight fit with the canister 42. Also integrally formed to the upper portion of the integral canister cap 44 is the integral thumb valve 56, and integral adapter 61 (integrally joined to integral thumb valve 56), as shown.

Preferably, the pneumatic medical system of FIG. 10 may be adapted to accommodate a wide range of standard (and under appropriate circumstances non-standard) sized medical canisters and specimen collection vials. The embodiment of FIG. 10 shows an integral canister cap 44, sized to fit a standard 125 mm (approximately ½ inch diameter) culture tube.

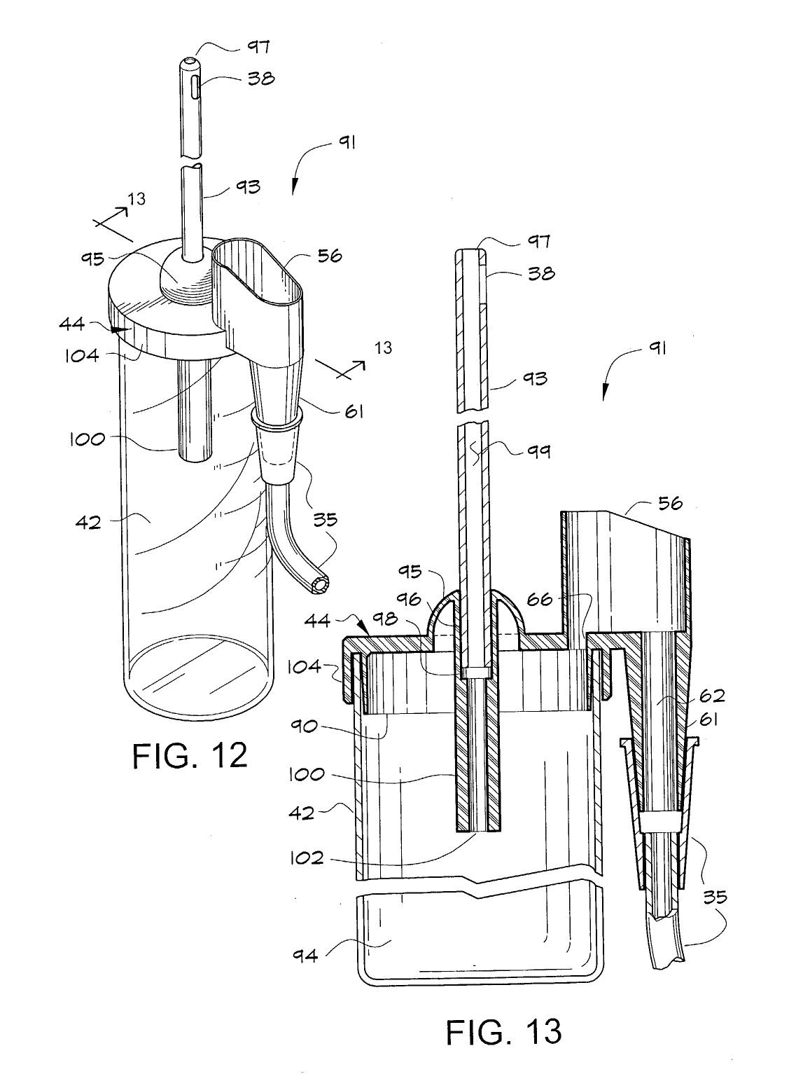

FIG. 11 is a sectional view through section 11-11 of FIG. 10 showing, in detail, the various interior assemblies of the pneumatic medical system. As described in FIG. 10, the fixed catheter 93, catheter connector 95 and canister 42 share the same approximate longitudinal central axis. Preferably, the interior of the catheter connecter 95 further comprises a catheter-receiving socket 96 (which also shares the same approximate longitudinal central axis), as shown. Preferably, a catheter stop 98 is molded into the base of the catheter-receiving socket 96, as shown. Preferably, extending down from the catheter stop 98 (through the internal hollow of the integral stopper 90) is at least one integrally molded sump extension 100, as shown. The integrally molded sump extension 100 serves as a return suction conduit between the fixed catheter 93 and the collection canister 42, as shown. In addition, the integrally molded sump extension 100 is preferably adapted to position the return suction inlet opening 102 away from the contents of the collection canister 42, should the pneumatic medical system be inverted. As previously discussed, in the field of suction devices with specimen collection canisters, it is desirable to prevent the collected specimen from occluding the return suction inlet to the container. Inverting a collection canister without a return suction inlet extension drains the collected specimen to the return suction inlet opening (then gravitationally located at the bottom of the inverted collection canister), thereby increasing the chances of inlet blockage. By preferably locating the return suction inlet opening 102 at the terminating end of the elongated integrally molded sump extension 100, the contents of the inverted canister are gravitationally held at the base of the inverted integrally molded sump extension 100, away from the return suction inlet opening 102.

Preferably, to maintain a compact size, portions of the integral thumb valve 56, integral canister cap 44 and integral catheter connector 95 share common structures, as show. Preferably, at least one portion of the integral thumb valve 56 is combined with the integral catheter connector 95, as shown. Preferably, the integral catheter connector 95 is adapted to include the vacuum port 66 that, as with the previous embodiments, allows air from the collection canister 42 to be drawn past the closed integral thumb valve 56, through the internal hollow space 62 of integral adapter 61 and out through outlet catheter 35.

FIG. 12 is a perspective view showing another preferred embodiment of the present invention utilizing a simplified integral canister cap 44, having a fixed catheter 93. The pneumatic medical system of FIG. 12 is preferably used with medium to large specimen collection canister(s) 42, as shown. Preferably, the pneumatic medical system comprises at least one fixed catheter 93, catheter connector 95, integral canister cap 44 (with integral stopper 90) and integrally molded sump extension 100, each sharing the same approximate longitudinal central axis of the collection canister 42, as shown. Preferably, at least one integral thumb valve 56 is integrally molded to the peripheral edge of the integral canister cap 44, as shown.

FIG. 13 is a sectional view through section 13-13 of FIG. 12 showing, in detail, the various interior assemblies of the pneumatic medical system. Preferably, the fixed catheter 93 is permanently (or under appropriate circumstances, removably) secured to the catheter connector 95, as shown. Preferably, the catheter connector 95 is integrally formed to the upper portion of the integral canister cap 44, as shown. Under appropriate circumstances the catheter connector 95 may be formed as a separate part that is threaded or friction fit to the integral canister cap 44, allowing a range of catheter sizes to be used with a single fixed catheter system 89. Preferably, the integral canister cap 44 of the large fixed catheter system 91 further comprises a peripheral flange 104 adapted to fit down and over the exterior of the canister 42, as shown. Peripheral flange 104 has a preferred height of about ¼ inch (0.64 cm). Preferably, at least one sump extension 100 is integrally molded to the lower portion of the catheter connector 95, as shown. Preferably, the integral canister cap 44 comprises at least one vacuum port 66, located within integral thumb valve 56 that, as with the previous embodiments, allows air from the collection canister 42 to be drawn past the closed integral thumb valve 56, through the internal hollow space 62 of integral adapter 61 and out through outlet catheter 35. Preferably, other functions and features of the large fixed catheter system 91 are substantially similar to the fixed catheter system 89 described in FIG. 10 and FIG. 11.

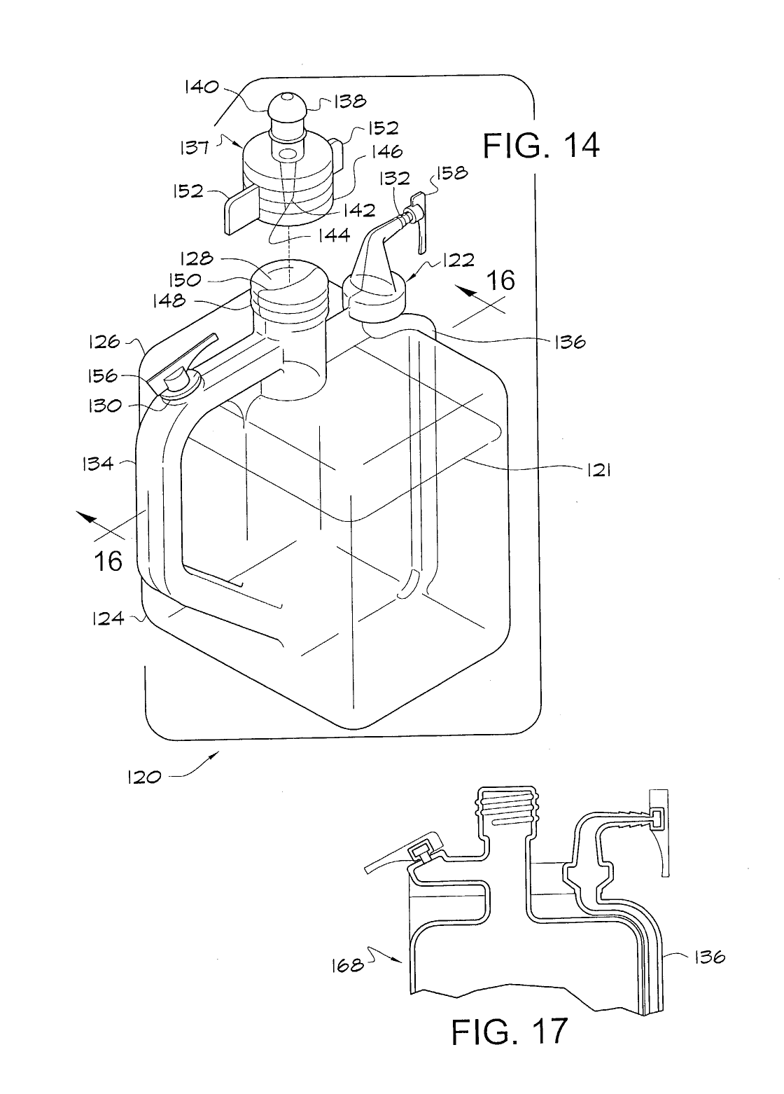

FIG. 14 is a perspective view of a disposable pre-filled irrigator 120 according to a preferred embodiment of the present invention. Preferably, the irrigator 120 comprises a delivery portion 122 and a reservoir 124, as shown. Preferably, the irrigator comprises a hermetically sealed container 126, preferably comprising a unitary structure, as shown. Preferably, the reservoir 124 contains a fluid, preferably an aqueous liquid 121, most preferably sterile, for use in irrigating wounds or other situations where a pressurized irrigation of sterile fluid is preferred. Those skilled in the art, upon reading the teachings of this specification, will appreciate that, under appropriate circumstances, considering such issues as medically prescribed or desired medications for irrigating and patient or husbandry usage, other types of liquid, such as an antibiotic additive or saline solution may suffice.

Preferably, the non-liquid components of irrigator 120 are formed from a thermoplastic material, such as polyethylene or polypropylene, using a multi-step blow-molding process. It is highly preferred that irrigator 120 be filled with a liquid and sealed while in the mold. This “blow-fill-seal” process is generally described in, for example, U.S. Pat. No. 4,187,951 to Cambio Jr., incorporated herein by reference as prior art enabling, in conjunction with this specification, the applicant's pre-filled irrigator.

Preferably, the delivery portion 122 comprises a sealed gas inlet 128, a removably sealed integral thumb valve 130 and a removably sealed fluid outlet 132, as shown. Preferably, a first walled chamber 134 connects the gas inlet 128 and integral thumb valve 130 to the reservoir 124, as shown. Preferably, a second walled chamber 136 connects the fluid outlet 132 to the reservoir 124, as shown. Preferably, the irrigator 120 further comprises a gas inlet cap 137, as shown. Preferably, the gas inlet cap 137 comprises a connector 138, preferably used to connect to an external pressurized gas source. Preferably, the connector 138 comprises an upper portion 140 structured to connect to tubing (such as described above in other pressurized embodiments) and a lower portion 142 comprising a piercing barb 144, as shown. Preferably, the gas inlet cap 137 has internal threads 146 that match external threads 148 on the sealed gas inlet 128 such that the gas inlet cap 137 may be screwed onto the sealed gas inlet 128 such that the piercing barb 144 will pierce the seal 150, as shown. Preferably, the gas inlet cap 137 also comprises tabs 152 for easy grabbing and twisting by a user, as shown. Those skilled in the art, upon reading the teachings of this specification, will appreciate that, under appropriate circumstances, considering such issues as economics and manufacturing, other methods of connecting and breaching the seal may suffice. For example, under appropriate circumstances, the gas inlet cap 137 may comprise a threaded coupler adapted to engage a wall mounted oxygen regulator valve (the mounting position being essentially similar to the wall-mounted arrangement described in, for example, U.S. Pat. No. 3,852,385 to Huggins, incorporated herein by reference). Under other appropriate circumstances, the sealed gas inlet 128 may preferably comprise an integral direct tubing connection having a tear-away seal (similar in structure and arrangement to the adjacent delivery portion 122).

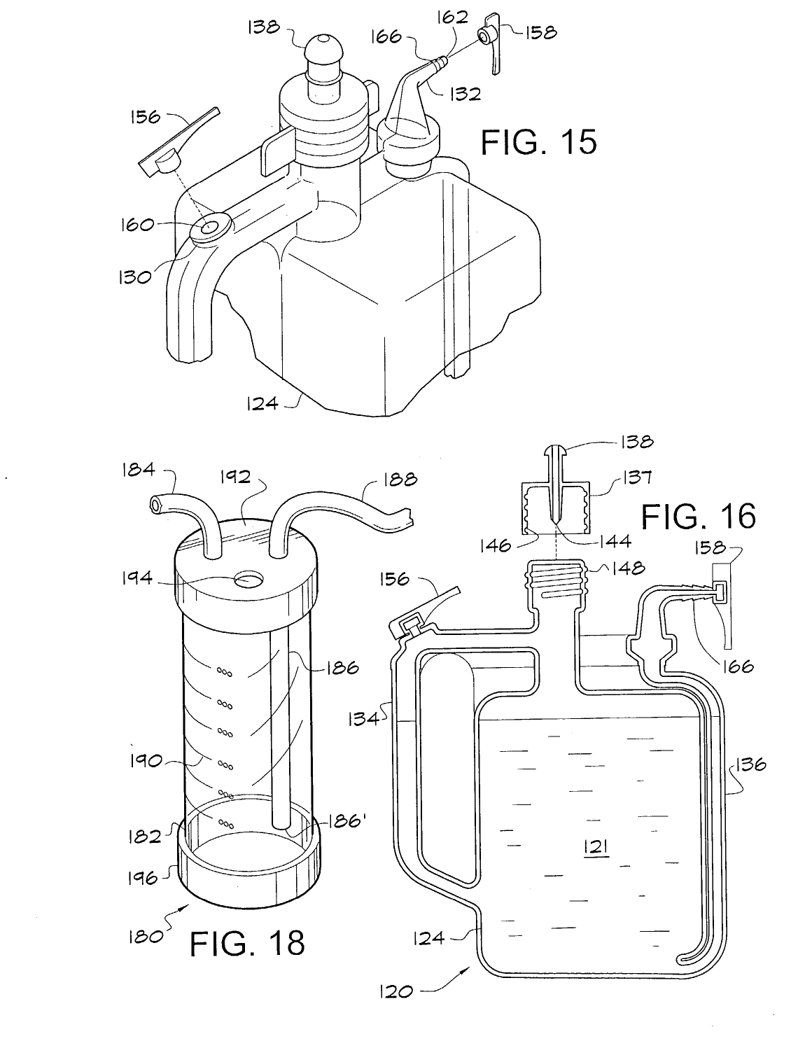

Reference is now made to FIG. 15 with continued reference to FIG. 14. FIG. 15 is a perspective view, partially in section of the upper portion 154 of the disposable pre-filled irrigator 120 of FIG. 14. Preferably, the integral thumb valve 130 and fluid outlet 132 comprise tear-away cap portion 156 and tear-away cap portion 158 that, when removed, unseal the respective sealed integral thumb valve 130 or fluid outlet 132 providing a respective opening 160 and opening 162, as shown.

Preferably, the connector 138 on the gas inlet cap 137 is sized such that tubing, preferably flexible tubing such as, for example, a tubing preferably approximately 70-85 durometer, preferably radiopaque, approximately 8 Fr (8 French) catheter tubing material or other such tubing that will hold the necessary pressure to pressurize the irrigator 120 is connected to the connector 138. Those skilled in the art, upon reading the teachings of this specification, will appreciate that, under appropriate circumstances, considering such issues as desired pressure, on-site equipment, standardized medical air lines and other desired uses for the irrigator such as use with an antibiotic additive or saline solution, other size tubing connections may suffice.

FIG. 16 is a sectional view through section 16-16 of FIG. 14. In the illustrated embodiment, when a pressurized line is connected to the connector 138, pressurized air preferably fills the reservoir 124. Preferably, if opening 160 on the integral thumb valve 130 is sealed (preferably by a thumb) and opening 162 is open, the pressurized air forces the liquid 121 in the reservoir 124 towards opening 162. Preferably, an outlet tubing or flushing catheter is attached over the fluid outlet 132. Preferably, such tubing attachment occurs over tapered barbed end 166. Those skilled in the art, upon reading the teachings of this specification, will appreciate that, under appropriate circumstances, considering such issues as tubing size and desired fluid output, other sizes of tubing or methods of attachment may suffice. Additionally, those skilled in the art, upon reading this specification, will appreciate that, under appropriate circumstances, such as during a high risk irrigation procedure, the use of a splash protector in conjunction with irrigator 120 (such as the optional splash shield 87 illustrated in FIG. 9), may suffice.

FIG. 17 is a sectional view of the upper portion 168 of the disposable pre-filled irrigator 120 illustrated in FIG. 16 illustrating another preferred embodiment of the disposable pre-filled irrigator 120. Preferably, in this embodiment, there is no first walled chamber 134, as shown above, only a walled chamber 170 (shown as second walled chamber 136 above), as shown.

FIG. 18 is a perspective view of an irrigation device according to a preferred embodiment of the present invention. Preferably, the irrigator 180 comprises a reservoir 182, an inlet line 184, an inner fluid outlet line portion 186 and an exterior fluid outlet line portion 188, as shown.

Preferably, the reservoir 182 comprises indicia 190, most preferably indicating volumetric measurement of the reservoir 182. Those skilled in the art, upon reading the teachings of this specification, will appreciate that, under appropriate circumstances, considering such issues as metric or English measurement, other methods of marking, such as ascending or descending markings may suffice.

Preferably, the reservoir comprises a top cap 192 and a bottom 196, as shown. Preferably, the top cap 192 comprises an aperture 194, preferably sized such that a user's thumb may easily block the aperture 194. Preferably, the inlet line portion 184 and exterior fluid outlet line portion 188 penetrated the top cap 192 and are sealably placed through such top cap 192. Those skilled in the art, upon reading the teachings of this specification, will appreciate that, under appropriate circumstances, considering such issues as manufacturing and economics, other methods of penetrating and sealing the inlet line portion 184 and exterior fluid outlet line portion 188, such as use of integral connectors or penetration of other portions of the irrigator 180, may suffice.

In operation, the inlet line 184 is pressurized such that any fluid (such as antibiotics etc.) is forced through the inner fluid outlet line portion 186 and out the exterior fluid outlet line portion 188. Preferably, by controlling the full or partial closing or opening of the aperture 194, a user can control the rate and degree of pressure forcing any fluid from the reservoir 182.

It is noted that irrigator 180 may also be used as a suction collection device simply by raising the end opening 186′ to a position near the top cap 192 while applying suction to the inlet line 184 rather than pressure, thereby drawing suction through the exterior fluid outlet line portion 188 and into the reservoir 182.

Although applicant has described applicant's preferred embodiments of this invention, it will be understood that the broadest scope of this invention includes such modifications as diverse shapes and sizes and materials. Such scope is limited only by the below claims as read in connection with the above specification. Further, many other advantages of applicant's invention will be apparent to those skilled in the art from the above descriptions and the below claims.

Further, for the purposes of this disclosure, the appended claims are to be deemed a part of the application specification as though fully set forth in the detailed descriptions.

What is claimed is:

1) A pneumatic medical system, usable in connection with a hand, having fingers and a thumb, comprising, in combination:
a) container means, graspable by the hand, for containing fluid;
b) first passage means and second passage means for defining a path of pressurized fluid through said container means;
c) modulator means for modulating, by use of the thumb, a force of the pressurized fluid through said container means;
d) integral cap means for capping said container means;
e) wherein said integral cap means comprises said modulator means.

2) The pneumatic medical system according to claim 1, further comprising:
a) restraining means for restraining at least one catheter;
b) wherein said integral cap means comprises said restraining means.

3) The pneumatic medical system according to claim 1, further comprising:
a) un-clogging means for un-clogging said container means;
b) wherein said integral cap means comprises said unclogging means.

4) A pneumatic medical system, usable in connection with a hand, having fingers and a thumb, comprising, in combination:
a) at least one container, graspable by the hand, adapted to contain fluid;
b) at least one first passage and at least one second passage structured and arranged to define a path of pressurized fluid through said at least one container;
c) at least one modulator adapted to modulate, by use of the thumb, a force of the pressurized fluid through said at least one container;
d) at least one integral cap adapted to cap said at least one container;
e) wherein said at least one integral cap comprises said at least one modulator.

5) The pneumatic medical system according to claim 4, further comprising:
a) at least one restrainer structured and arranged to restrain at least one catheter;
b) wherein said at least one integral cap comprises said at least one restrainer.

6) The pneumatic medical system according to claim 5, further comprising:
a) at least one clip;
b) wherein said at least one restrainer comprises said at least one clip.

7) The pneumatic medical system according to claim 4, further comprising:
a) at least one un-clogger adapted to un-clog said at least one container;
b) wherein said at least one integral cap comprises said at least one un-clogger.

8) The pneumatic medical system according to claim 7, further comprising:
a) at least one removable replacement lid;
b) wherein said at least one container comprises indicia structured and arranged to assist in determining an amount of substance within said at least one container; and
c) wherein said at least one modulator comprises at least one finger valve.

9) The pneumatic medical system according to claim 4, wherein said at least one modulator comprises at least one finger valve.

10) The pneumatic medical system according to claim 4, further comprising:
a) at least one endotracheal tube adaptor port;
b) wherein said at least one integral cap comprises said at least one endotracheal tube adaptor port.

11) The pneumatic medical system according to claim 4, wherein:
a) said at least one container comprises indicia structured and arranged to assist in determining an amount of substance within said at least one container.

12) The pneumatic medical system according to claim 4, wherein:
a) said at least one container comprises advertising indicia.

13) The pneumatic medical system according to claim 4, further comprising:
a) at least one removable replacement lid.

14) The pneumatic medical system according to claim 4, further comprising:
a) at least one smaller-diameter catheter;
b) at least one larger-diameter catheter structured and arranged to fit into at least one said passage;
c) wherein said at least one smaller-diameter catheter and said at least one larger-diameter catheter are structured and arranged so that said at least one smaller-diameter catheter is connectable to said at least one larger-diameter catheter by being pulled through said at least one larger-diameter catheter.

15) The pneumatic medical system according to claim 14, wherein said at least one smaller-diameter catheter comprises at least one side hole.

16) The pneumatic medical system according to claim 4, wherein said at least one container has a substantially vertical central longitudinal axis.

17) The pneumatic medical system according to claim 16, wherein said at least one container comprises substantially a right circular cylinder.

18) A pneumatic medical device, usable in connection with a hand, having fingers and a thumb, for use with at least one container having a substantially vertical central longitudinal axis and structured and arranged to contain a fluid, comprising:
a) at least one integral cap, structured and arranged to cap the at least one container, and comprising:
i) at least one first passage and at least one second passage, structured and arranged, with the at least one container, to define a path of pressurized fluid through the at least one container; and
ii) at least one modulator adapted to modulate, by use of the fingers or thumb, a force of the pressurized fluid through said at least one container.

19) The pneumatic medical device according to claim 18, wherein said at least one integral cap further comprises at least one restrainer structured and arranged to restrain at least one catheter.

20) The pneumatic medical device according to claim 18, wherein said at least one modulator comprises at least one finger valve.

21) The pneumatic medical device according to claim 18, wherein said at least one integral cap further comprises at least one endotracheal tube adaptor port.

22) The pneumatic medical device according to claim 19, further comprising:
a) at least one clip;
b) wherein said at least one restrainer comprises said at least one clip.

23) The pneumatic medical device according to claim 18, wherein said at least one integral cap further comprises at least one un-clogger adapted to un-clog the container.

24) The pneumatic medical device according to claim 18, wherein:
a) at least one said passage comprises at least one downwardly-extending passage extension; and
b) at least one said passage comprises said at least one modulator.

25) The pneumatic medical device according to claim 18, further comprising:
a) at least one smaller-diameter catheter;
b) at least one larger-diameter catheter structured and arranged to fit into at least one said passage;
c) wherein said at least one smaller-diameter catheter and said at least one larger-diameter catheter are structured and arranged so that said at least one smaller-diameter catheter is connectable to said at least one larger-diameter catheter by being pulled through said at least one larger-diameter catheter.

26) The pneumatic medical device according to claim 25, wherein said at least one smaller-diameter catheter comprises at least one side hole.

27) The pneumatic medical device according to claim 18, wherein the longitudinal axes of said first passage, said second passage, and the container, are essentially parallel.

28) The pneumatic medical device according to claim 18, further comprising:
a) at least one connector structured and arranged to connect at least one of at least one first passage and at least one second passage to at least one pressurized fluid source.

29) The pneumatic medical device according to claim 28, wherein;
a) at least one adaptor structured and arranged to attach at least one of said at least one first passage and at least one second passage to at least one fluid splash shield.

30) The pneumatic medical device according to claim 29, wherein said at least one adaptor comprises at least one luer adaptor.

31) A pneumatic medical system comprising:
a) at least one smaller-diameter catheter; and
b) at least one larger-diameter catheter;
c) wherein at least one of said at least one smaller-diameter catheter and said at least one larger-diameter catheter comprises at least one flexible catheter;
d) wherein said at least one smaller-diameter catheter is releasably connected, via an airtight frictional fit, within said at least one larger-diameter catheter;
e) wherein said at least one smaller-diameter catheter and said at least one larger-diameter catheter each comprise a proximal and a distal end; and
f) wherein said distal end of said at least one smaller-diameter catheter extends beyond said distal end of said at least one larger-diameter catheter.

32) A pneumatic medical system comprising, in combination:
a) at least one source of oxygen under pressure;
b) at least one container adapted to contain irrigation fluid;
c) wherein said at least one container comprises at least one inlet adapted to inlet such oxygen under pressure from said at least one source into an interior upper portion of said at least one container; and
d) wherein said at least one container comprises at least one outlet adapted to outlet such irrigation fluid from an interior lower portion of said at least one container; and
e) at least one connector adapted to connect said at least one source with said at least one container in such manner as to assist forming a passageway for such oxygen under pressure from said at least one source to enter said at least one inlet;
f) wherein, when said at least one container contains such irrigation fluid and when such oxygen under pressure enters said at least one container, such irrigation fluid will expel through said at least one outlet and thus be available for irrigation use.

33) The pneumatic medical system according to claim 32 further comprising a splash shield system having a fluid connection with said at least one outlet.

34) A pneumatic medical system comprising the steps of:
a) providing a sealed container of irrigation fluid, such container comprising at least one unsealable inlet and at least one unsealable outlet;
b) unsealing such container by unsealing such at least one unsealable inlet and such at least one unsealable outlet;
c) connecting such unsealed at least one unsealable inlet with a source of oxygen under pressure; and
d) using the irrigation fluid expelled from such container when oxygen enters such unsealed at least one unsealable inlet for irrigation.
